(19) United States
(12) Patent Application Publication
(10) Pub. No.: US 2018/0141498 A1
(43) Pub. Date: May 24, 2018

(54) VEHICLE SUN VISOR MOUNT FOR AIR SENSOR DEVICES

(71) Applicant: Jesse Jake Savage, Boca Raton, FL (US)

(72) Inventor: Jesse Jake Savage, Boca Raton, FL (US)

(21) Appl. No.: 15/356,628

(22) Filed: Nov. 20, 2016

(51) Int. Cl. B60R 11/00; G01N 33/00

(52) U.S. Cl. CPC B60R 11/00; B60R 2011/0035; G01N 33/004

(57) ABSTRACT

A sun visor sensor mount has an elongate planar base with an arcuate biased clip configured for compression fit of the device to a sun visor of a vehicle. The device has a rectangular casing for securely retaining a bendable card with an air sensor device such as a carbon monoxide sensor. One or more lips extend into the opening of the casing to hold a bendable card within the casing in place. A bendable card may be inserted or removed by flexing the card. An aperture in the back of the casing may be used to insert a finger or other device to push a bendable card out of the casing.

### STATEMENT REGARDING FEDERALLY SPONSORED RESEARCH OR DEVELOPMENT

Not Applicable.

### THE NAMES OF PARTIES TO A JOINT RESEARCH AGREEMENT

Not Applicable

### INCORPORATION-BY-REFERENCE OF THE MATERIAL SUBMITTED ON A COMPACT DISC

Not Applicable.

### COPYRIGHT NOTICE

Not Applicable

### BACKGROUND OF THE INVENTION

### Field of the Invention

The present invention relates to a sun visor sensor mount. More particularly, the invention relates to a device for securing a sensor to a sun visor of an automobile or other vehicle.

### Description of the Related Art

Historically, air sensors have been installed inside a vehicle, such as for example cars, trucks, aircraft and the like, using tape, 2-sided tape, hook-and-loop (Velcro®) fasteners, magnets and other methods of attaching them to dashboards, windows and panels. This often requires the use of tape, glue, adhesives or other chemicals that can damage or discolor dashboards, windows and panels when they are removed or replaced. Furthermore, these forms of installation do not allow an air sensor to be easily moved from vehicle to vehicle.

Air sensors are completely susceptible to many elements in the environment—sunlight/UV, large air particles, pollutants, humidity, liquids, aerosols, tampering, touching, abuse, etc. . . . drastically reducing their effectiveness and their useful operational life.

The above-described deficiencies of today's systems are merely intended to provide an overview of some of the problems of conventional systems, and are not intended to be exhaustive. Other problems with the state of the art and corresponding benefits of some of the various non-limiting embodiments may become further apparent upon review of the following detailed description.

In view of the foregoing, it is desirable to provide a sun visor mount for a sensor on a bendable card.

### BRIEF SUMMARY OF THE INVENTION

Disclosed is a sun visor mount for a sensor that keeps the sensor near the roofline—away from most airborne contaminants and nearer to the warmer and lighter gasses. This places a sensor outside of the general area of activity for passengers, reducing the risk of inadvertent damage. It is also not exposed to sunlight, is out of the way of air vents and other objects inside a vehicle. This allows an air sensor to operate more effectively, accurately and for a much longer period of time.

In one embodiment, the mount is adapted for use with sensor cards, for example bendable sensor cards, which may be easily interchanged or replaced without specialized tools or removing the mount itself. The mount itself is easily transferred from one vehicle to another without specialized tools or damage to the vehicles.

In one embodiment, a sun visor sensor mount comprises an elongate, substantially planar base, an arcuate biased clip extending from the distal end of the base, and a bendable card retention casing. The casing has a back panel coextensive with a portion of the planar base and two or more opposing walls extending from the planar base. The casing includes an aperture in the back panel. A bendable card is secured within the retention casing by at least one lip extending over a front opening of the casing. The sun visor sensor mount may also comprise a bendable card within the casing.

It is therefore an object of the present invention to provide a sun visor mount for a sensor on a bendable card.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### BRIEF DESCRIPTION OF THE SEVERAL VIEWS OF THE DRAWINGS

A more complete understanding of the present invention, and the attendant advantages and features thereof, will be more readily understood by reference to the following detailed description when considered in conjunction with the accompanying drawings wherein:

FIG. 1 is a perspective view of a sun visor sensor mount in accordance with the principles of the invention;

FIG. 2 is a top plan view of a sun visor sensor mount in accordance with the principles of the invention;

FIG. 3 is a cross-sectional view of a sun visor sensor mount along plane A of FIG. 2 in accordance with the principles of the invention;

FIG. 4 is a side elevation view of a sun visor sensor mount in accordance with principles of the invention;

FIG. 5 is a bottom perspective view of a sun visor sensor mount in accordance with the principles of the invention;

FIG. 6 is a side view of an alternative embodiment of a sun visor sensor mount in accordance with the principles of the invention;

FIG. 7 is a top plan view of an alternative embodiment of a sun visor sensor mount in accordance with the principles of the invention;

FIG. 8 is a side elevation view of an alternative embodiment of a sun visor sensor mount in accordance with the principles of the invention.

### DETAILED DESCRIPTION OF THE INVENTION

The invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The disclosed subject matter is described with reference to the drawings, wherein like reference numerals are used to refer to like elements throughout. In the following description, for purposes of explanation, numerous specific details are set forth in order to provide a thorough understanding of the various embodiments of the subject disclosure. It may be evident, however, that the disclosed subject matter may be practiced without these specific details. As used herein, the words “top” and “upward” refer to a same general direction evident in the drawings, and refer to the side and direction of the device opposite to the side or direction of a sun visor upon which the device is removably affixed, and in the same direction in which the sensor card casing is positioned relative to the planar base of the device. Similarly, the words “bottom” and “downward” also refer to directions made clear in the drawings.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

Disclosed is a sun visor mount for a bendable card in accordance with the present invention that keeps the sensor near the roofline—away from most airborne contaminants and nearer to the warmer and lighter gasses. The sun visor sensor mount of the present invention has a planar base. A biased clip extends from and folds over the back of the base and is capable of holding the mount securely to an object such as a sun visor by means of a friction fit between the clip and the back of the base. The front of the base includes a retention casing for a bendable card.

The retention casing of one embodiment consists of four orthogonal walls extending upward from the base to form a parallelepiped chamber in which a bendable card is retained by the casing. The casing thus extends over five of the six sides of a bendable card. The top of the casing is an opening. One or more of the orthogonal walls may have a lip extending partially over the opening to securely retain the bendable card within the casing.

In one embodiment, the sun visor sensor mount is formed from a material that is substantially rigid, and allows a bendable card to snap in and out of the casing over a small lip extending from the top of one or more of the walls of the casing. The sun visor sensor mount may optionally include an aperture through the base to assist in the removal of a sensor retained within the casing.

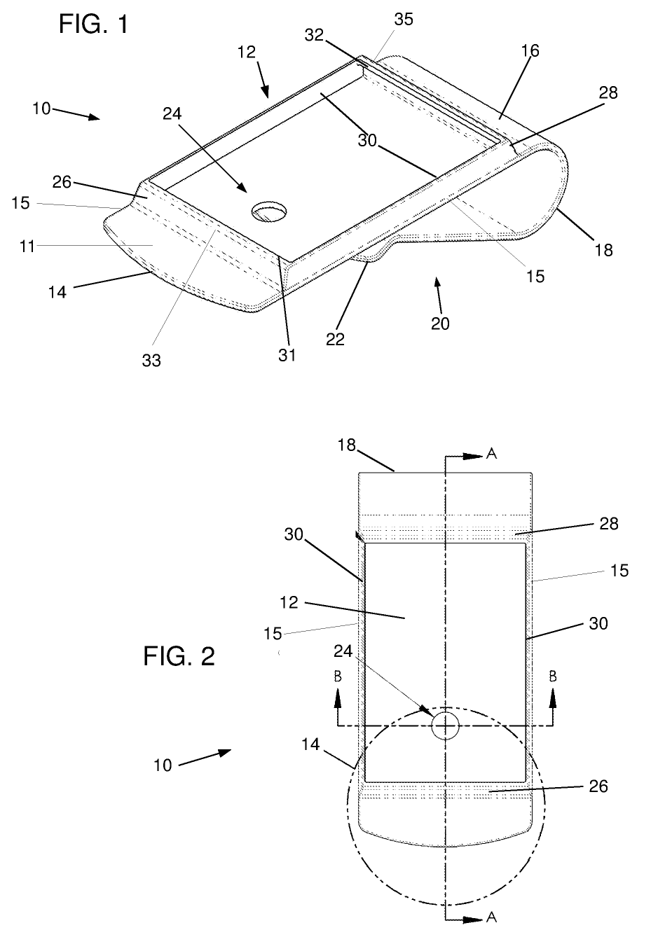

FIGS. 1-5 show a sun visor sensor mount 10 in accordance with the principles of the invention. The sun visor sensor mount 10 has a substantially planar base 12 having a top side 11 and a bottom side 13. The planar base 12 extends between a proximal end 14 to a distal end 16. The planar base 12 of this embodiment is further defined by two lateral sides 15. A clip member 20 is attached to the distal end 16 of the planar base 12. The arcuate region 18 of the clip member 20 extends outward from the distal end 16 and curves more than 180°. The sun visor sensor mount 10 may be comprised of a sturdy and rigid material that provides some flexibility such that the mounts may form a friction fit attachment between the base 12 and clip 20 around a sun visor of a vehicle. PVC and other plastics as well as sheet-metal may all have suitable properties.

Those skilled in the art will appreciate that such clips are known and used for removably attaching garage door remote controls and similar devices to sun visors. The distal end 22 of the clip member 20 may curve outward to facilitate separating the clip 20 from the base 12 so that it may be placed over a sun visor or other device. The sun visor sensor mount 10 is thus removably affixed to a sun visor by means of a friction fit generated by the bias toward the base of the clip member 20.

The sun visor sensor mount 10 includes a proximal wall 26 and a distal wall 28, both protruding upward from the base 12 on the top side 11 of the base 12. In this embodiment, two lateral walls 30 also protrude from the base 12 on the lateral sides 15 and extend between the proximal wall 26 and the distal wall 28. The proximal wall 26, the distal wall 28, the two lateral walls 30 and the portion of the base 12 defined by the protruding walls form a casing 34 that retains a bendable card, not shown. Proximal lip 31 extends in a distal direction from the top 33 of the proximal wall 26. Similarly, a distal lip 32 extends in a proximal direction from the top 35 of the distal wall 28. Both the proximal lip 31 and the distal lip 32 extend partially into the opening in the casing defined by the protruding walls 26, 28 and 30, thereby securing in place a sensor positioned within the casing.

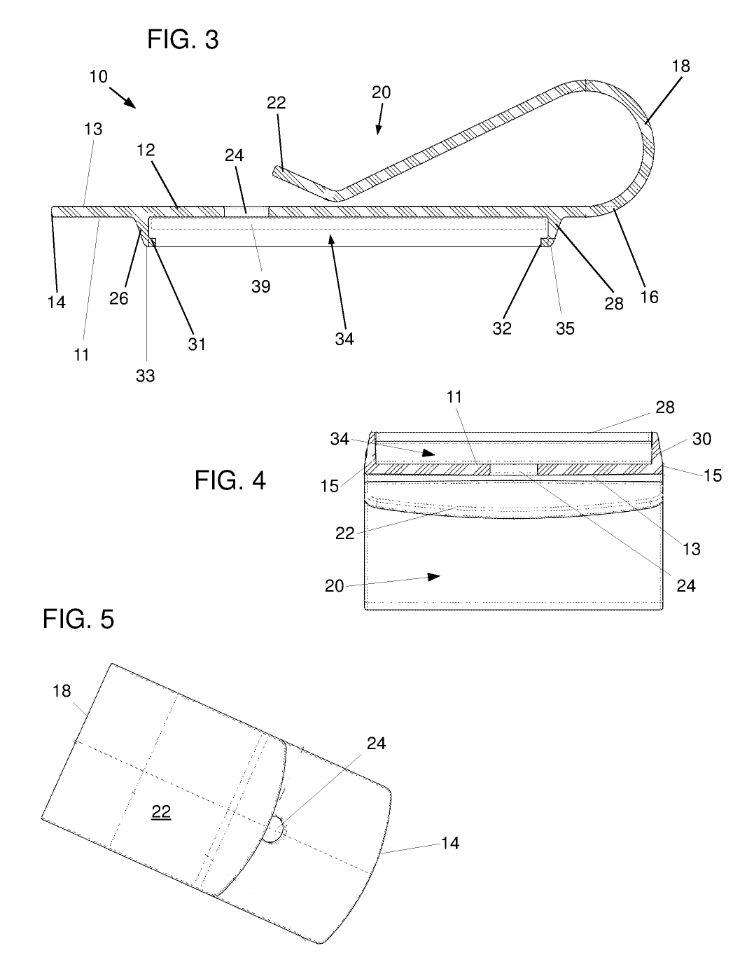

FIG. 3 shows the sun visor sensor mount 10 in an inverted orientation. In this Figure, an outline shows the location of a bendable card sensor 39 when it is removably inserted into the casing 34. The aperture 24 in the base 12 allows an operator to push the back of a sensor retained within the casing 34 to facilitate removal and/or replacement of the sensor.

Optionally, the lateral walls 30 may not be included in the sun visor sensor mount. A bendable card may be held in place only by the proximal wall and the distal wall. Optionally, the lateral walls 30, the proximal wall 26 and a distal wall 28 may be only partial walls, may include slips, apertures or openings in them and thus not be solid walls. Optionally, a bendable card may be retained on the sun visor sensor mount by means of magnets, adhesive, tape, Velcro® and/or ferromagnetic material on the mounts on the sensor and a complementary corresponding position. Optionally, only one of the proximal wall and the distal wall may have a lip extending into the opening of the casing. Optionally, the lips extending into the opening of the casing may be rubber or other elastic material to facilitate bending or stretching of the lips to allow insertion and removal of a bendable card.

Optionally, instead of using a clip to retain the sun visor sensor mount on a sun visor, the sun visor sensor mount may replace the clip with one or more elastic bands, Velcro®, laces, a spring biased clip, adhesives, combinations of these various mechanisms, or the like. Optionally, grip tape or a friction enhancing coating may be placed on one or both of the clip and the base of the sun visor sensor mount.

Optionally, an air sensor device may be integrated into the mount such that they are a single unitary device. Optionally, there may be no aperture in the base. Optionally, the aperture may be filled with a deformable material such as rubber. Optionally, a logo, trademark or other ornamentation or design may be placed on the sun visor sensor mount, on a portion of the base such as for example the proximal region of the base, or on the clip.

In one embodiment, the bendable card is inserted into the opening of the casing by slightly bending the card to allow it to fit between the proximal lip and the distal lip. The bendable card is then placed within the casing. Once the card is no longer bent, the lips extending from the proximal and distal walls secure the bendable card within the casing. To remove the bendable card, the card is again bent to increase the distance between the proximal and distal lips. A finger or other device is then inserted through the aperture to exert force on the back of the bendable card.

The bending of the card can also be done from the rear. A removal/ejection hole can be accessed from the underside of the sun visor mount. The user can use a finger, pen/pencil or small rod to press the bendable card out from under the two undercuts, thereby freeing the bendable card from the sun visor sensor mount.

In one embodiment, the sun visor sensor mount is formed from a black nylon with UV inhibitors. Optionally, other materials may be used, and in particular materials that inhibit UV and other harmful electromagnetic waves and/or are water resistant. The sun visor sensor mount may be manufactured using injection molding. Optionally, the bendable card is, for example a CR80 (credit card) sized plastic card, typically made of PVC or PVC composites.

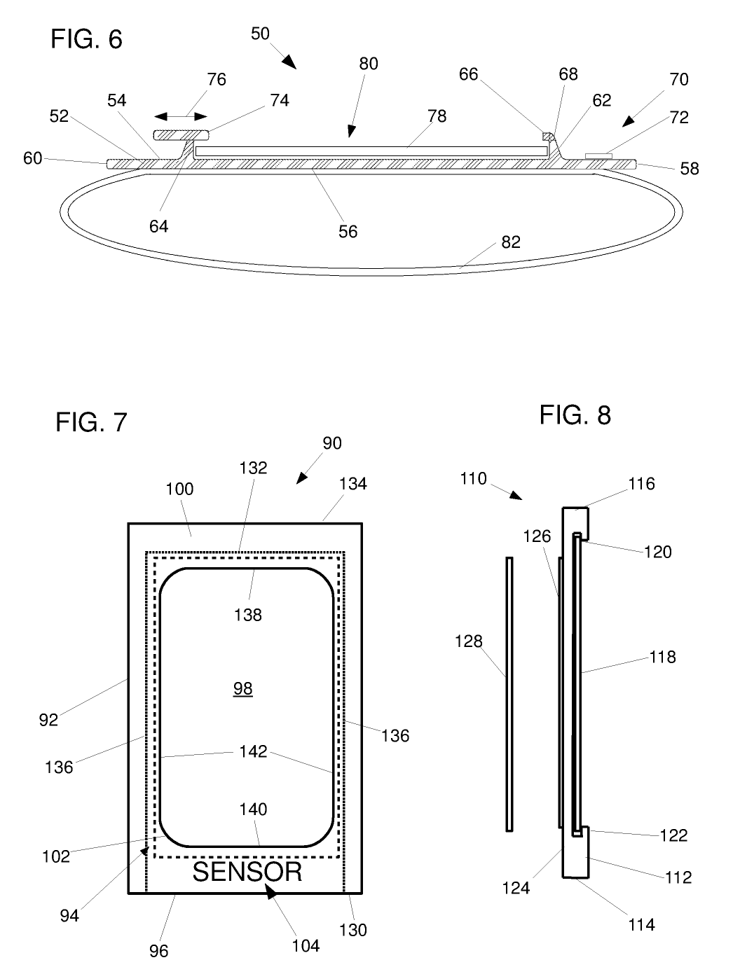

FIG. 6 shows an alternative embodiment of a sun visor sensor mount 50 in accordance with the principles of the invention. Sun visor sensor mount 50 includes a planar base 52 having a top side 54 and a bottom side 56. The planar base 52 extends from a proximal end 58 to a distal end 60. A proximal wall 62 extends upward from the top side 54 of the base 52. A distal wall 64 similarly extends upward from the top side 54 of the base 52 and is parallel to the proximal wall 62. A proximal lip 66 extends in a distal direction from the top 68 of the proximal wall 62. The proximal region 70 of this embodiment includes an embossed ornamentation 72.

Distal wall 64 of this embodiment includes a sliding lip 74 that slides in both proximal and distal directions as indicated by arrow 76. When the sliding lip 74 is slid in a distal direction, sensor card 78 is inserted into the casing cavity 80 defined by the top side 54 of the base 52, the proximal wall 62 and the distal wall 64. When the sliding lip 74 is slid in the proximal direction, the sensor card 78 is secured within the casing cavity 80. The sliding lip 74 may be biased by a spring or other mechanism in the proximal direction.

An elastic band 82 may be affixed to the bottom side 56 of the base 52. In use, the sun visor sensor mount 50 may be affixed to a sun visor or other object by wrapping the elastic band 82 about the object to removably secured in place. The elastic band 82 may optionally be replaced with laces, Velcro straps or the like.

FIG. 7 shows an alternative embodiment of a sun visor sensor mount 90 in accordance with the principles of the invention. Sun visor sensor mount 90 has a planar base 92. The base 92 includes sensor card retention case 94 accessible through an opening 96. A sensor card 98 may be inserted into the sensor card retention case 94 through the opening 96 on the distal end 130 of the base 92. In this embodiment, the sensor card retention case 94 is defined by a proximal wall 132 on the proximal end 134 and two lateral sidewalls 136. In this embodiment, there is no distal wall so that a distal opening 96 may be used for inserting and removing the sensor card 98. The sensor card 98 within the sensor card retention case 94 is visible from the top side 100 through a window 102. The window 102 is defined by a proximal lip 138 extending in a distal direction, a distal lip 140 extending in a proximal direction, and two lateral lips 142 extending toward each other. The bottom side, not shown, of the base 92 may have a clip member, and elastic band or other device for removable fixation on to a sun visor. The top side 100 may also include ornamentation 104.

FIG. 8 shows an alternative embodiment of a sun visor sensor mount 110 in accordance with the principles of the invention. Sun visor sensor mount 110 includes a planar base 112 defined by a proximal end 114 and a distal end 116. A sensor card 118 may be slid into an elongate groove extending transversely across the base 112. A distal lip 120 and a proximal lip 122 hold the sensor card 118 securely in place by means of a friction fit. The bottom side 124 of the base 92 includes an adhesive pad 126. The adhesive pad may include an adhesive for attachment to a sun visor. Optionally, the adhesive pad may be configured to removably engage and affixed to an adhesive pad 128 that may be affixed to a sun visor or other device. For example, the adhesive pads 126 and 128 may be comprised of Velcro® or a similar complementary affixation mechanism.

In use, a sun visor sensor mount may be removably affixed to a sun visor in a vehicle and a bendable card having a sensor may be inserted into it. Because of its location, a sun visor sensor mount positions the bendable card and sensor in a location where it is less likely to be exposed to conditions that impact the accuracy of the sensor. For example, a sensor located on a dashboard is exposed to sunlight, heat and debris settling on the dashboard. A sensor located on one of the air vents in a vehicle is exposed to the conditions of air circulated in the vehicle. In other words, the sensor may experience additional heat or cold. A sensor mounted on a console is more likely to be jarred, displaced, scratched and exposed to other conditions that may affect its accuracy. By placing a sensor on a sun visor that is kept out of the sunlight, the sensor renders more accurate readings. In addition, it is positions in a location readily viewable by operators and passengers in a vehicle.

Whereas, the present invention has been described in relation to the drawings attached hereto, it should be understood that other and further modifications, apart from those shown or suggested herein, may be made within the spirit and scope of this invention. Descriptions of the embodiments shown in the drawings should not be construed as limiting or defining the ordinary and plain meanings of the terms of the claims unless such is explicitly indicated.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

1. A sun visor sensor mount comprising:
a planar base having a top side and a bottom side, and defined by a proximal end, a distal end and two sides;
an attachment means for removably affixing the sun visor sensor mount;
a rectangular card casing on the top side of the base, the casing defined by a back panel coextensive with a portion of the planar base, a proximal wall extending upward from the top side of the base and an opposing distal wall extending upward from the top side of the base;
a proximal retention channel defined by the back panel, the proximal wall and a proximal lip extending in a distal direction from the proximal wall;
a distal retention channel defined by the back panel, the distal wall and a distal lip extending in a proximal direction from the distal wall.

2. The sun visor sensor mount of claim 1 wherein the attachment means comprises a clip member facing the bottom side of the base and attached to the base by an arcuate region extending from the distal end of the base;

3. The sun visor sensor mount of claim 1 further comprising a card removably secured in the rectangular card casing, the card having a proximal end positioned within the proximal retention channel and a distal end positioned within the distal retention channel.

4. The sun visor sensor mount of claim 3 wherein the card includes a carbon monoxide sensor.

5. The sun visor sensor mount of claim 1 wherein the rectangular card casing is further defined by two opposing lateral walls extending upward from the sides of the base.

6. The sun visor sensor mount of claim 1 further comprising an aperture in the back panel of the bendable card retention casing.

7. The sun visor sensor mount of claim 1 wherein one of the proximal lip and the distal lip is slidable.

8. The sun visor sensor mount of claim 1 further comprising ornamentation on the top side of the base proximal to the proximal end of the base.

9. The sun visor sensor mount of claim 1 wherein the sun visor sensor mount is removably affixed to a sun visor of a vehicle.

10. The sun visor sensor mount of claim 2 further comprising:
a card removably secured in the rectangular card casing, the card having a proximal end positioned within the proximal retention channel and a distal end positioned within the distal retention channel;
an aperture in the back panel of the bendable card retention casing; and,
ornamentation on the top side of the base proximal to the proximal end of the base.
wherein the rectangular card casing is further defined by two opposing lateral walls extending upward from the sides of the base; and,
wherein one of the proximal lip and the distal lip is slidable.

11. The sun visor sensor mount of claim 1 wherein the attachment means comprises an elastic band affixed to the back side of the base.

12. The sun visor sensor mount of claim 11 wherein the rectangular card casing is further defined by two opposing lateral walls extending upward from the sides of the base.

13. The sun visor sensor mount of claim 12 further comprising an aperture in the back panel of the bendable card retention casing;

14. The sun visor sensor mount of claim 13 further comprising ornamentation on the top side of the base proximal to the proximal end of the base

15. The sun visor sensor mount of claim 14 wherein the sun visor sensor mount is removably affixed to a sun visor of a vehicle.

16. A method of detecting carbon monoxide comprising:
providing a sun visor sensor mount comprising a planar base having a top side and a bottom side, and defined by a proximal end, a distal end and two sides; an attachment means for removably affixing the sun visor sensor mount; a rectangular card casing on the top side of the base, the casing defined by a back panel coextensive with a portion of the planar base, a proximal wall extending upward from the top side of the base and an opposing distal wall extending upward from the top side of the base; a proximal retention channel defined by the back panel, the proximal wall and a proximal lip extending in a distal direction from the proximal wall; a distal retention channel defined by the back panel, the distal wall and a distal lip extending in a proximal direction from the distal wall;
inserting a bendable card having a carbon monoxide sensor into the rectangular card casing of the sun visor sensor mount;
affixing the sun visor sensor mount to a sun visor in a vehicle;
visually monitoring the carbon monoxide sensor while in the vehicle.
